(19) United States
(12) Patent Application Publication
(10) Pub. No.: US 2009/0105718 A1
(43) Pub. Date: Apr. 23, 2009

(54) SURGICAL INSTRUMENTS FOR USE WITH BREAK-OFF DEVICE AND AN ASSOICATED SURGICAL METHOD

(75) Inventors: Jeffrey ZHANG, Collierville, TN (US); John D. POND, Germantown, TN (US); Randy N. ALLARD, Germantown, TN (US); Larry T. MCBRIDE, Memphis, TN (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC , INC., Warsaw, IN (US)

(21) Appl. No.: 11/873,558

(22) Filed: Oct. 17, 2007

(51) Int. Cl. A61B 17/88

(52) U.S. Cl. 606/104

(57) ABSTRACT

A surgical instrument for use with a break-off device including a proximal portion removably attached to a distal portion by a region of reduced strength to allow selective removal of the proximal portion from the distal portion. The surgical instrument includes an inner drive shaft extending generally along a longitudinal axis and having a distal end portion engaged with the proximal portion of the break-off device, and an outer retention sleeve positioned about the drive shaft and including a distal receiver portion engaged about the proximal portion of the break-off device. A force applied to the proximal portion of the break-off device by the drive shaft causes the break-off device to fracture at the region of reduced strength to separate the proximal portion from the distal portion, with the proximal portion maintained in engagement with the surgical instrument by the retention sleeve.

### FIELD OF THE INVENTION

The present invention relates generally to the field of surgical instrumentation, and more particularly relates to a surgical instrument for use with a break-off device and an associated surgical method.

### BACKGROUND

Various types of implants are commonly used in the treatment of anatomic structures such as the spinal column. In the spinal field, implants are sometimes used which include devices wherein a portion of the device is broken off and removed from the remainder of the implant. Such devices include, for example, set screws having an upper head portion that is broken off and removed from a lower threaded body portion of the set screw. Due care must be taken by the surgeon to maintain engagement with the portion of the device which is broken away from the implant to ensure removal from the patient's body. This can be particularly difficult in minimally invasive surgical procedures where visualization of the implant and/or the surgical site may be partially or totally obstructed.

Thus, there remains a need for an improved surgical instrument for use with a break-off device and an associated surgical method. The present invention satisfies this need and provides other benefits and advantages in a novel and unobvious manner.

### SUMMARY

The present invention relates generally to a surgical instrument for use with a break-off device and an associated surgical method. While the actual nature of the invention covered herein can only be determined with reference to the claims appended hereto, certain forms of the invention that are characteristic of the preferred embodiments disclosed herein are described briefly as follows.

In one form of the present invention, a surgical instrument is provided for use in association with a break-off device including a proximal portion removably attached to a distal portion by a region of reduced strength to allow selective removal of the proximal portion from the distal portion, with the surgical instrument including an inner drive shaft extending generally along a longitudinal axis and including a distal end portion engaged with the proximal portion of the break-off device, and an outer retention sleeve positioned about the drive shaft and including a distal receiver portion engaged about the proximal portion of the break-off device. A force applied to the proximal portion of the break-off device by the drive shaft causes the break-off device to fracture at the region of reduced strength to separate the proximal portion from the distal portion, with the proximal portion maintained in engagement with the surgical instrument by the retention sleeve.

In another form of the present invention, surgical instrumentation is provided including a break-off set screw having a proximal head portion removably attached to an externally threaded distal body portion by a region of reduced strength to allow selective removal of the proximal head portion from the distal body portion. The surgical instrumentation further includes a driver instrument including an inner drive shaft extending generally along a longitudinal axis and an outer retention sleeve positioned about the inner drive shaft, with the drive shaft including a distal end portion engaged with the proximal head portion of the break-off set screw, and with the retention sleeve including a distal receiver portion engaged about the proximal head portion of the break-off set screw. A force applied to the proximal head portion of the break-off set screw by the drive shaft causes the break-off set screw to fracture at the region of reduced strength to separate the proximal head portion from the distal body portion, with the proximal head portion maintained in engagement with the surgical instrument by the retention sleeve.

In a further form of the present invention, a surgical method is provided including providing a break-off device having a proximal portion removably attached to a distal portion by a region of reduced strength to allow selective removal of the proximal portion from the distal portion, providing a surgical instrument including an inner drive shaft extending generally along a longitudinal axis and an outer retention sleeve positioned about the inner drive shaft, engaging a distal portion of the drive shaft with the proximal portion of the break-off device, engaging a distal receiver portion of the retention sleeve about the proximal portion of the break-off device, applying a force to the proximal portion of the break-off device via the drive shaft to fracture the break-off device at the region of reduced strength, separating the proximal portion of the break-off device from the distal portion, and retaining the proximal portion of the break-off device on the surgical instrument by maintaining engagement of the receiver portion of the retention sleeve about the proximal portion of the break-off device.

It is one object of the present invention to provide an improved surgical instrument for use with a break-off device and an associated surgical method. Further objects, features, advantages, benefits, and aspects of the present invention will become apparent from the drawings and description contained herein.

### BRIEF DESCRIPTION OF THE DRAWINGS

FIG. 1 is a side view of a surgical instrument according to one form of the present invention.

FIG. 2 is a cross sectional side view of a distal portion of the surgical instrument shown in FIG. 1.

FIG. 3 is an enlarged cross sectional side view of the distal portion of the surgical instrument shown in FIG. 1.

FIG. 4 is an end view of the surgical instrument shown in FIG. 1.

FIG. 5 is a side view of a break-off device according to one embodiment of the present invention for use with the surgical instrument shown in FIG. 1.

FIG. 6 is a cross sectional side view of the break-off device shown in FIG. 5.

FIG. 7 is a side view of the distal portion of the surgical instrument shown in FIG. 1, as engaged with the break-off device shown in FIG. 5.

FIG. 8 is a cross sectional side view of the distal portion of the surgical instrument engaged with the break-off device, as taken along line 8-8 of FIG. 7, and as positioned within an outer cannula tube.

### DESCRIPTION OF THE PREFERRED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is hereby intended, and that alterations and further modifications to the illustrated devices and/or further applications of the principles of the invention as illustrated herein are contemplated as would normally occur to one skilled in the art to which the invention relates.

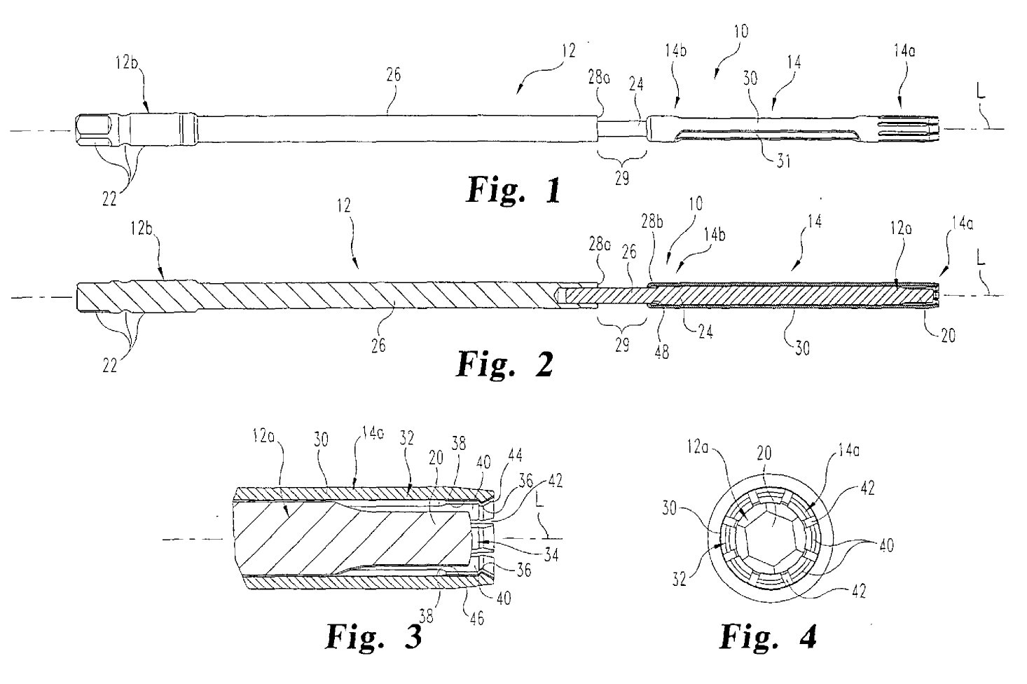

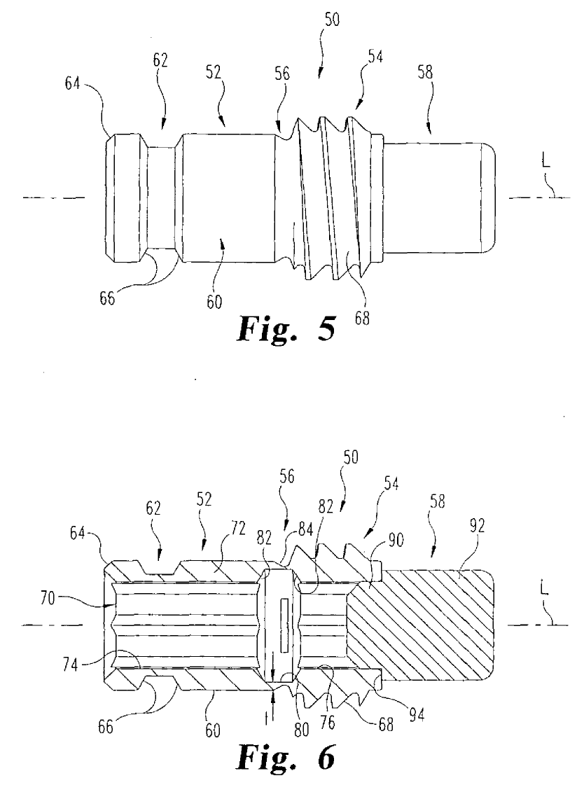

Referring to FIGS. 1-4, shown therein is a surgical instrument 10 according to one form of the present invention. In the illustrated embodiment, the surgical instrument 10 is configured as a driver instrument for use in association with a break-off device, such as, for example, the break-off device 50 illustrated in FIGS. 5 and 6. The driver instrument 10 extends along a longitudinal axis L and is generally comprised of an inner shaft or shank 12 and an outer sleeve 14 positioned about the inner shaft 12. In the illustrated embodiment, the driver instrument 10 is configured such that the outer sleeve 14 is axially displaceable along the inner shaft 12, the details of which will be set forth below. As will also be set forth below, the inner shaft 12 includes a distal end configured for driving engagement with the break-off device, and the outer sleeve 14 is configured to extend about a proximal portion of the break-off device to retain the proximal portion in engagement with the instrument 10 subsequent to being broken off from the remainder of the device. The components of the driver instrument 10 and the break-off device are formed of biocompatible materials including, for example, metallic materials such as stainless steel or titanium, and/or polymeric materials such as PEEK. However, other biocompatible materials are also contemplated including, for example, metallic alloy materials, reinforced composite materials, or any other suitable biocompatible material that would occur to one of ordinary skill in the art.

The inner drive shaft 12 extends along the longitudinal axis L and includes a distal portion 12a and a proximal portion 12b. The distal portion 12a of the drive shaft 12 includes a distal end portion 20 configured for driving engagement with a proximal portion of the break-off device. In the illustrated embodiment, the distal end portion 20 is configured as an axially-extending projection or stem sized and shaped for driving engagement within an axial passage formed in the proximal portion of the break-off device. However, other configurations of the distal end portion 20 are also contemplated. For example, the distal end portion 20 could alternatively be provided with a recess or socket sized and configured to engagingly receive a projection or stem extending proximally from the break-off device. Additionally, in the illustrated embodiment, the distal stem portion 20 has a hexagonal shape. However, other shapes and configurations are also contemplated, including a Torx™ style shape, a star shape, a cross shape, a rectangular shape, a triangular shape, other polygonal shapes, or any other suitable shape or configuration that would occur to one of ordinary skill in the art. The proximal portion 12b of the drive shaft 12 includes features 22 configured for engagement with a handle or drive mechanism. In the illustrated embodiment, the drive shaft 12 is formed from two axial segments, including a distal segment 24 and a proximal segment 26 which are operatively interconnected to provide an integral shaft assembly. However, a unitary, single-piece drive shaft is also contemplated. In one embodiment, the distal and proximal segments 24, 26 each have a generally circular outer cross-section, with the distal segment 24 having an outer cross-section sized smaller than the outer cross-section of the proximal segment 26 to define a distally-facing shoulder 28a. Additionally, the distal segment 24 includes a reduced cross-sectional region which defines a proximally-facing shoulder 28b. The shoulders 28a, 28b in turn define an axially-extending annular groove 29 along a length of the drive shaft 12, the purpose of which will be discussed below.

The outer retention sleeve 14 extends along the longitudinal axis L and includes a distal portion 14a and a proximal portion 14b. In the illustrated embodiment, the retention sleeve 14 has a tubular configuration defined by a cylindrical-shaped side wall 30 which may be provided with a number of openings 31 extending through the side wall 30 and communicating with the interior of the retention sleeve 14. One purpose of the openings 31 is to facilitate sterilization of the inner surfaces of the retention sleeve 14 and the portion of the drive shaft 12 positioned within the retention sleeve 14. Another purpose of the openings 31 is to reduce the weight of the surgical instrument 10. In the illustrated embodiment, the openings 31 are provided as elongated slots extending generally along the longitudinal axis L. However, it should be understood that other types and configuration of openings are also contemplated.

The distal portion 14a of the retention sleeve 14 includes a receiver portion 32 defining an axial passage 34 sized and shaped to receive a proximal portion of the break-off device therein. In the illustrated embodiment, the receiver portion 32 includes a transverse projection 36 extending inwardly into the axial passage 34 and configured for releasable engagement within a groove or recess formed along an exterior surface of the proximal portion of the break-off device. In one embodiment, the transverse projection 36 is configured as an annular lip or ring extending about an interior surface 38 of the receiver portion 32. However, it should be understood that the annular lip 36 need not necessarily extend entirely about the inner perimeter of the receiver portion 32, but may instead extend partially about the inner perimeter and/or may be peripherally interrupted. Additionally, although the receiver portion 32 is illustrated as including a single transverse projection or lip 36, it should be understood that the receiver portion 32 may define a two or more transverse projections or lips which are circumferentially and/or axially offset from one another. In one embodiment, the annular lip 36 is provided with an arcuate or rounded profile. However, other shapes and configurations are also contemplated.

In the illustrated embodiment, the receiver portion 32 is provided with a plurality of flexibly resilient arms or fingers 40 extending generally along and positioned about the longitudinal axis L. The flexible arms 40 are formed via a number of axially-extending slots or slits 42 defined through the side wall 30 of the retention sleeve 40 and extending proximally from the distal end of the receiver portion 32. The flexible arms 40 are transitionable between an initial state and an outwardly expanded state. In one embodiment, the flexible arms 40 are positioned in the initial state when in a relaxed or non-stressed condition, and are transitioned or deflected to the outwardly expanded state via the imposition of an outward deformation force onto the flexible arms 40 during insertion of the proximal portion of the break-off device into the axial passage 34 of the receiver portion 32. Once the break-off device is positioned within the receiver portion 32, the flexible arms 40 are inwardly biased to exert a compression force onto the proximal portion of the break-off device to maintain engagement of the retention sleeve 14 with the break-off device. Engagement of the retention sleeve 14 with the break-off device is facilitated by positioning of the annular lip 36 within an annular groove formed about the break-off device, and/or frictional engagement between inner surfaces of the flexible arms 40 and an outer surface of the break-off device.

As illustrated in FIG. 3, the distal end of the receiver portion 32 may be provided with a tapered inner surface 44 to aid in insertion of the proximal portion of the break-off device into the axial passage 34 of receiver portion 32 and to facilitate outward deformation of the flexible fingers 40. Additionally, the distal end of the receiver portion 32 may be provided with a tapered or chamfered outer surface 46 to minimize trauma or injury to adjacent tissue. Furthermore, as illustrated in FIG. 2, the proximal portion 14b of the retention sleeve 14 may be provided with an inwardly extending annular flange or lip 48 which is positioned within the annular groove 29 defined by the drive shaft 12 to capture the retention sleeve 14 on the drive shaft 12 while permitting relative axial displacement of the retention sleeve 14 along the drive shaft 12, the purpose of which will be discussed below.

Referring now to FIGS. 5 and 6, shown therein is one embodiment of a break-off device 50 for use in association with the driver instrument 10. The break-off device 50 generally includes a proximal portion 52 removably attached to a distal portion 54 by a region of reduced strength 56 to allow selective removal of the proximal portion 52 from the distal portion 54. In the illustrated embodiment, the break-off device 50 comprises a break-off set screw, with the proximal portion 52 configured as a head portion, and the distal portion 54 configured as an externally threaded body portion. The head portion 52 is connected to the body portion 54 by the region of reduced strength 56 which permits the head portion 52 to be selectively broken off and removed from the body portion 54, the details of which will be set forth below. The break-off set screw 50 may optionally include a distal plug member 58 engaged with and extending from the body portion 54. Although the break-off device 50 has been illustrated and described as a break-off set screw, it should be understood that the other types and configurations of break-off devices are also contemplated for use in association with the driver instrument 10 including, for example, other types of fasteners, bolts, pins, posts, or any other type of break-off device that would occur to one of skill in the art.

The head portion 52 of the break-off set screw 50 is sized and configured for receipt within the axial passage 34 defined by the receiver portion 32 of the retention sleeve 14 to engage the set screw 50 with the driver instrument 10. In one embodiment, the head portion 52 includes an exterior peripheral surface 60 defining a generally circular outer cross section. However, other shapes and configurations of the head portion 52 are also contemplated. In the illustrated embodiment, the exterior surface 60 of the head portion 52 defines a recess or notch 62 sized and configured to receive the transverse projection or lip 36 defined by the receiver portion 32 of the retention sleeve 14 to selectively and releasably engage the retention sleeve 14 to the head portion 52 of the break-off set screw 50. In the illustrated embodiment, the recess 62 is configured as an annular groove extending about the head portion 52. However, it should be understood that the annular groove 62 need not necessarily extend entirely about the head portion 52. It should further be understood that in another embodiment, the head portion 52 may alternatively define an outwardly extending transverse projection which is received within a recess defined by the receiver portion 32 of the retention sleeve 14 to selectively and releasably engage the retention sleeve 14 with the head portion 52 of the break-off set screw 50. Additionally, the proximal end of the head portion 52 may be provided with a chamfer 64 to facilitate insertion of the head portion 52 into the axial passage 34 of the receiver portion 32. The end walls 66 of the annular groove 62 may be angled or chamfered to facilitate insertion and removal of the annular lip 36 of the receiver portion 32 into and out of the annular groove 62.

In one embodiment, the body portion 54 of the break-off set screw 50 includes external threads 68 configured for threading engagement with an internally threaded passage defined by an implant or another device. Such implants or device may include, for example, various types and configurations of connectors, plates, interbody devices, or any other implant or device that would occur to one of skill in the art. In the illustrated embodiment, the external threads 68 are configured as machine threads. However, other types and configurations of threads are also contemplated. For example, in another embodiment of the invention, the break-off device 50 may be configured as a bone screw, including a proximal head portion and a distal body portion defining external threads configured for engagement within bone tissue, such as cancellous bone tissue.

In the illustrated embodiment, the break-off set screw 50 defines an axial passage 70 extending at least partially therethrough, and a side wall 72 extending peripherally about and bounding the axial passage 70. In one embodiment, the axial passage 70 extends entirely through the break-off set screw 50 from the proximal end of the head portion 52 to the distal end of the body portion 54, thereby providing the break-off set screw 50 with a cylindrical configuration. However, it should be understood that the axial passage 70 may alternatively extend partially through the break-off set screw 50. As shown in FIG. 6, the portion of the axial passage 70 extending through the head portion 52 defines a tool-receiving socket or recess 74 sized and shaped to drivingly receive the distal end portion 20 of the drive shaft 12 therein to allow the body portion 54 of the break-off set screw 50 to be driven into a threaded opening in an implant or another device, and/or to exert a force onto the head portion 52 to break off the head portion 52 from the body portion 54. Additionally, the portion of the axial passage 70 extending through the body portion 54 also defines a tool-receiving socket or recess 76 sized and shaped to receive the distal end portion 20 of the drive shaft 12 therein (or a distal end portion of another tool or instrument) to allow the body portion 54 of the break-off set screw 50 to be driven into a threaded opening in an implant or another device, and/or to allow for removal of the body portion 54 from the threaded opening following separation of the head portion 52 from the body portion 54. In the illustrated embodiment, the tool-receiving sockets 74 and 76 each have a hexagonal shape. However, other shapes and configurations are also contemplated, including a Torx™ style shape, a star shape, a cross shape, a rectangular shape, a triangular shape, other polygonal shapes, or any other suitable shape or configuration that would occur to one of ordinary skill in the art.

In the illustrated embodiment of the break-off set screw 50, the portion of the axial passage 70 extending along the region of reduced strength 56 defines an undercut section 80. The undercut section 80 provides the axial passage 70 with an enlarged region, which correspondingly provides the side wall 72 with a reduced cross-sectional thickness t adjacent the region of reduced strength 56. The undercut section 80 may be provided with angled walls 82 extending inwardly to the inner walls of the sockets 74 and 76. Additionally, the exterior surface 60 of the break-off set screw 50 may define an annular groove or channel 84 extending peripherally about the head portion 52, which further reduces the cross-sectional thickness t adjacent the region of reduced strength 56. In the illustrated embodiment, the annular groove 84 has an arcuate or curved configuration. However, other shapes and configurations of the annular groove 84 are also contemplated. As should be appreciated, providing the break-off set screw 50 with the undercut section 80 and/or the annular groove or channel 84 reduces the cross-sectional thickness t of the side wall 70, which correspondingly provides the region of reduced strength 56. As should further be appreciated, the region of reduced strength 56 provides a weakened area between the head portion 52 and the body portion 54 such that application of a force onto the head portion 52 causes the region of reduced strength 56 to fracture to allow the head portion 52 to be broken off and separated from the body portion 54.

As indicated above, the break-off set screw 50 may optionally include a distal plug member 58 engaged with and extending from the body portion 54. In one embodiment, the plug member 58 is formed of a polymeric material, such as, for example, PEEK. However, other suitable materials are also contemplated, including elastomeric, metallic or composite materials. In the illustrated embodiment, the distal plug member 58 includes a proximal portion 90 and a distal portion 92. The proximal portion 90 is positioned within the socket 76 defined by the body portion 54 of the break-off set screw 50. In one embodiment, the proximal portion 90 has a hexagonal shape which corresponds to the hexagonal shape of the socket 76. In a further embodiment, the proximal portion 90 is sized to frictionally engage the proximal portion 90 within the socket 76 to retain the plug member 58 in engagement with the body portion 54 of the break-off set screw 50. The distal portion 92 has a generally circular outer cross section, and the proximal portion 90 has an outer cross section sized somewhat smaller than the distal portion 92 to provide an axially-facing shoulder 94 which abuts the distal end of the body portion 54 to properly position the plug member 58 relative to the body portion 54 and to prevent the plug member 58 from being inserted too far into the socket 76. Although the proximal and distal portions 90, 92 of the plug member 58 are illustrated and described as having specific shapes and configurations, it should be understood that other shapes and configurations are also contemplated.

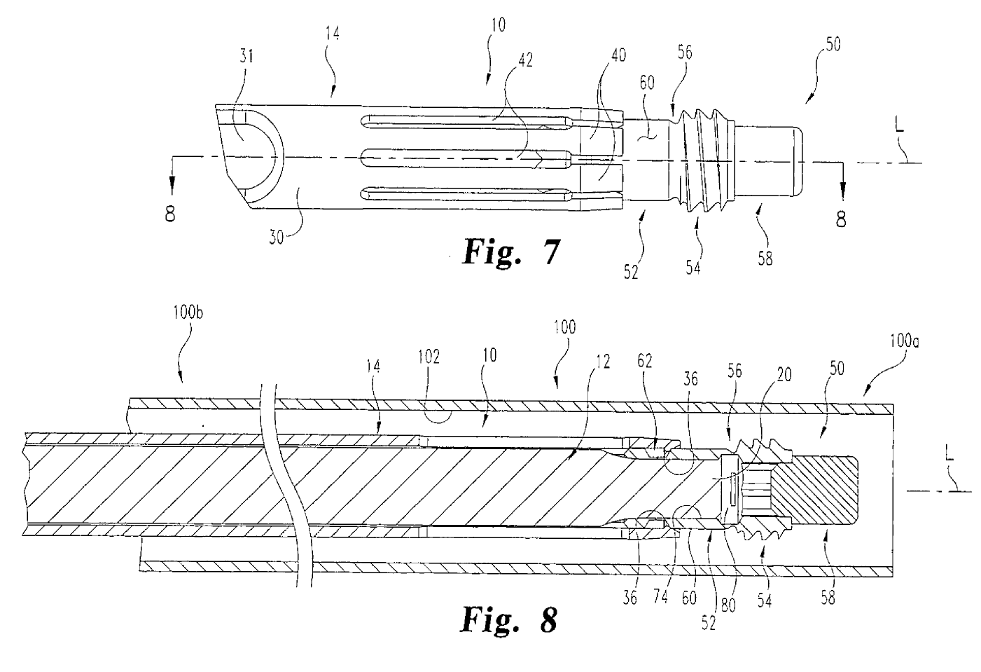

Referring to FIGS. 7 and 8, shown therein is the driver instrument 10 engaged with the break-off set screw 50. The driver instrument 10 is initially engaged with the break-off set screw 50 by sliding the retention sleeve 14 in a proximal direction relative to the drive shaft 12 to expose the distal end portion 20 of the drive shaft 12. The distal end portion 20 is then inserted into the tool-receiving socket 74 defined by the head portion 52 of the break-off set screw 50. The retention sleeve 14 in then displaced in a distal direction relative to the drive shaft 12, and the receiver portion 32 is slid over the head portion 52 of the break-off set screw 50. The flexible fingers 40 of the receiver portion 32 are outwardly expanded as the head portion 52 is slid into the axial passage 34 of the receiver portion 32. Further displacement of the retention sleeve 14 positions the annular lip 36 of the receiver portion 32 within the annular groove 62 defined by the head portion 52, which in turn causes the flexible fingers 40 to inwardly contract about the head portion 52 to capture the head portion 52 within the receiver portion 32 of the retention sleeve 14. The break-off set screw 50 is then delivered to the surgical site and the threaded body portion 54 is driven into a threaded opening in an implant or another device. A force is then applied to the head portion 52 of the break-off set screw 50 via the drive shaft 12 to fracture the break-off set screw 50 at the region of reduced strength 56 to separate the head portion 52 from the body portion 54, with the head portion 52 captured within the receiver portion 32 of the retention sleeve 14 to maintain engagement of the head portion 52 with the surgical instrument 10. The head portion 52 may then be safely removed from the surgical site without risk of dropping the head portion 52 into the patient. In one embodiment of the invention, the force applied to the head portion 52 by the drive shaft 12 to facilitate a fracture at the region of reduced strength 56 is a rotational or torsional force which twists the head portion 52 relative to the body portion 54. However, it should be understood that other forces applied to the drive shaft 12 may be used to fracture the region of reduced strength 56, including a lateral bending or shearing force.

Following removal of the head portion 52 from the patient, the head portion 52 is disengaged from the driver instrument 10 by axially displacing the retention sleeve 14 in a proximal direction relative to the drive shaft 12 to slide the receiver portion 32 off of the head portion 52. As should be appreciated, sliding the retention sleeve 14 in a proximal direction along the drive shaft 12 causes the flexible fingers 40 of the receiver portion 32 to outwardly expand to allow removal/disengagement of the annular lip 36 from the annular groove 62 defined by the head portion 52. Once the head portion 52 is removed from the axial passage 34 of the receiver portion 32, the head portion 52 can be slid off of the distal end portion 20 of the drive shaft 12 and discarded. Another break-off set screw 50 may then be engaged to the driver instrument 10 for delivery to the surgical site.

As illustrated in FIG. 8, in a further embodiment of the invention, a cannula tube 100 may be used to deliver the break-off set screw 50 to a location adjacent the surgical site via a minimally invasive surgical procedure. The cannula tube 100 includes a distal end 100a and a proximal end 100b, and defines an axial passage or protected channel 102 extending threrethrough between the distal and proximal ends 100a, 100b. The cannula tube 100 also has a length l such that the proximal end 100b is disposed outside of a patient's body when the distal end 100a is positioned adjacent a surgical site. The axial passage 102 is sized to receive the driver instrument 10 and the break-off set screw 50 therethrough such that the break-off set screw 50 may be delivered through the cannula tube 100 to a location adjacent the surgical site for engagement with an implant or another device. The break-off set screw 50 may be percutaneously delivered to a location adjacent the surgical site in a minimally invasive manner via the protected channel 102 provided by the cannula tube 100. Additionally, following removal of the proximal head portion 52 from the threaded body portion 54 of the break-off set screw 50, the head portion 52 is maintained in engagement with the surgical instrument 10 so that the head portion 52 can be safely removed from the surgical site via the cannula tube 100.

While the invention has been illustrated and described in detail in the drawings and foregoing description, the same is to be considered as illustrative and not restrictive in character, it being understood that only the preferred embodiments have been shown and described and that all changes and modifications that come within the spirit of the invention are desired to be protected.

What is claimed is:

1. A surgical instrument for use in association with a break-off device including a proximal portion removably attached to a distal portion by a region of reduced strength to allow selective removal of the proximal portion from the distal portion, the surgical instrument comprising:
an inner drive shaft extending generally along a longitudinal axis and including a distal end portion engaged with the proximal portion of the break-off device; and
an outer retention sleeve positioned about the drive shaft and including a distal receiver portion engaged about the proximal portion of the break-off device; and
wherein a force applied to the proximal portion of the break-off device by the drive shaft causes the break-off device to fracture at the region of reduced strength to separate the proximal portion from the distal portion with the proximal portion maintained in engagement with the surgical instrument by the retention sleeve.

2. The surgical instrument of claim 1, wherein the proximal portion of the break-off device defines one of a transverse projection and a recess, and with the receiver portion of the retention sleeve defining another of the transverse projection and the recess; and
wherein the transverse projection is received within the recess to releasably engage the retention sleeve to the proximal head portion of the break-off set device.

3. The surgical instrument of claim 2, wherein the recess comprises an annular groove extending about an exterior surface of the proximal portion of the break-off device, and
wherein the transverse projection comprises an annular lip extending about an interior surface of the receiver portion of the retention sleeve.

4. The surgical instrument of claim 2, wherein the receiver portion of the retention sleeve includes a plurality of flexible arms extending generally along the longitudinal axis and being transitionable between an initial state and an outwardly expanded state, the flexible arms transitioned to the expanded state to receive the proximal portion of the break-off device within the receiver portion and transitioned back toward the initial state to position the transverse projection within the recess to securely engage the break-off device with the surgical instrument.

5. The surgical instrument of claim 1, wherein the receiver portion of the retention sleeve includes a plurality of flexible arms extending generally along the longitudinal axis and being transitionable between an initial state and an outwardly expanded state, the flexible arms transitioned to the expanded state to receive the proximal portion of the break-off device within the receiver portion and transitioned back toward the initial state to engage the flexible arms with the proximal portion of the break-off device.

6. The surgical instrument of claim 1, wherein the drive shaft includes an exterior surface defining an axially-extending groove; and
wherein the retention sleeve includes an inwardly extending projection positioned within the axially-extending groove to capture the retention sleeve on the drive shaft while permitting relative axial displacement of the retention sleeve along the drive shaft via axial displacement of the inwardly extending projection within the axially-extending groove.

7. Surgical instrumentation, comprising:
a break-off set screw including a proximal head portion removably attached to an externally threaded distal body portion by a region of reduced strength to allow selective removal of the proximal head portion from the distal body portion; and
a driver instrument including an inner drive shaft extending generally along a longitudinal axis and an outer retention sleeve positioned about the inner drive shaft, the drive shaft including a distal end portion engaged with the proximal head portion of the break-off set screw, the retention sleeve including a distal receiver portion engaged about the proximal head portion of the break-off set screw; and
wherein a force applied to the proximal head portion of the break-off set screw by the drive shaft causes the break-off set screw to fracture at the region of reduced strength to separate the proximal head portion from the distal body portion with the proximal head portion maintained in engagement with the surgical instrument by the retention sleeve.

8. The surgical instrumentation of claim 7, wherein the proximal portion of the break-off set screw defines one of a transverse projection and a recess, and with the receiver portion of the retention sleeve defining another of the transverse projection and the recess; and
wherein the transverse projection is received within the recess to releasably engage the retention sleeve to the proximal head portion of the break-off set screw.

9. The surgical instrumentation of claim 8, wherein the proximal head portion of the break-off set screw includes an exterior surface defining the recess; and
wherein the receiver portion of the retention sleeve defines the transverse projection extending inwardly from a side wall of the receiver portion.

10. The surgical instrumentation of claim 8, wherein the receiver portion of the retention sleeve includes a plurality of flexible arms extending generally along the longitudinal axis and being transitionable between an initial state and an outwardly expanded state, the flexible arms transitioned to the expanded state to receive the proximal head portion of the break-off set screw within the receiver portion and transitioned back toward the initial state to position the transverse projection within the recess to securely engage the break-off set screw with the driver instrument.

11. The surgical instrumentation of claim 8, wherein the recess comprises an annular groove.

12. The surgical instrumentation of claim 11, wherein the transverse projection comprises an annular lip.

13. The surgical instrumentation of claim 7, wherein the receiver portion of the retention sleeve includes a plurality of flexible arms extending generally along the longitudinal axis and being transitionable between an initial state and an outwardly expanded state, the flexible arms transitioned to the expanded state to receive the proximal head portion of the break-off set screw within the receiver portion and transitioned back toward the initial state to engage the flexible arms with the proximal head portion of the break-off set screw.

14. The surgical instrumentation of claim 7, wherein the break-off set screw defines a passage extending at least partially therethrough and providing the proximal head portion with a tool-receiving aperture, the drive shaft including a distal stem portion received within the tool-receiving aperture; and
wherein the passage is bound by a side wall of the break-off set screw with the region of reduced strength defined by a reduced thickness of the side wall.

15. The surgical instrumentation of claim 14, wherein the reduced thickness of the side wall is at least partially defined by an annular groove extending about the exterior surface of the proximal head portion of the break-off set screw.

16. The surgical instrumentation of claim 15, wherein the receiver portion of the retention sleeve includes an inwardly extending transverse projection received within the annular groove to releasably engage the retention sleeve with the proximal head portion of the break-off set screw.

17. The surgical instrumentation of claim 14, wherein the passage defined by the proximal head portion of the break-off set screw extends entirely through the break-off set screw to provide each of the proximal head portion and the distal body portion with a tool-receiving aperture.

18. The surgical instrumentation of claim 7, wherein the drive shaft includes an exterior surface defining an annular groove having a width extending along the longitudinal axis; and
wherein the retention sleeve includes an inwardly extending flange positioned within the annular groove to capture the retention sleeve on the drive shaft while permitting relative axial displacement of the retention sleeve along the drive shaft.

19. The surgical instrumentation of claim 7, further comprising a cannula tube including a distal end and a proximal end, the cannula tube defining an axial passage extending threrethrough between the distal end and the proximal end; and
wherein the cannula tube has a length such that the proximal end is disposed outside of a patient's body when the distal end is positioned adjacent a surgical site, the axial passage sized to receive the driver instrument therethrough to deliver the break-off set screw to a location adjacent the surgical site.

20. A surgical method, comprising:
providing a break-off device including a proximal portion removably attached to a distal portion by a region of reduced strength to allow selective removal of the proximal portion from the distal portion, and providing a surgical instrument including an inner drive shaft extending generally along a longitudinal axis and an outer retention sleeve positioned about the inner drive shaft;
engaging a distal end portion of the drive shaft with the proximal portion of the break-off device;
engaging a distal receiver portion of the retention sleeve about the proximal portion of the break-off device;
delivering the break-off device to a surgical site;
applying a force to the proximal portion of the break-off device via the drive shaft to fracture the break-off device at the region of reduced strength;
separating the proximal portion of the break-off device from the distal portion;
retaining the proximal portion of the break-off device on the surgical instrument by maintaining engagement of the receiver portion of the retention sleeve about the proximal portion of the break-off device; and
removing the proximal portion of the break-off device from the surgical site.

21. The surgical method of claim 20, wherein the proximal portion of the break-off device defines one of a transverse projection and a recess, and with the receiver portion of the retention sleeve defining another of the transverse projection and the recess; and
wherein the engaging of the receiver portion about the proximal portion of the break-off device comprises positioning of the transverse projection within the recess.

22. The surgical method of claim 21, wherein the recess comprises an annular groove extending at least partially about an exterior surface of the proximal portion of the break-off device, and wherein the inwardly extending transverse projection comprises an annular lip extending about an interior surface of the receiver portion of the retention sleeve.

23. The surgical method of claim 21, wherein the receiver portion of the retention sleeve includes a plurality of flexible arms extending generally along the longitudinal axis and being transitionable between an initial state and an outwardly expanded state, the flexible arms transitioned to the expanded state to receive the proximal portion of the break-off device within the receiver portion; and
wherein the engaging of the receiver portion about the proximal portion of the break-off device comprises transitioning of the flexible fingers back toward the initial state to position the transverse projection within the recess to securely engage the break-off device with the surgical instrument.

24. The surgical method of claim 20, wherein the drive shaft includes an annular groove having a width extending along the longitudinal axis, and wherein the retention sleeve includes an inwardly extending flange positioned within the annular groove to capture the retention sleeve on the drive shaft; and
further comprising axially displacing the retention sleeve along the drive shaft to disengage the proximal portion of the break-off device from the receiver portion of the retention sleeve.

25. The surgical method of claim 20, wherein the break-off device comprises a break-off set screw, and wherein the proximal portion comprises a head portion and the distal portion comprise an externally threaded body portion; and
further comprising engaging the externally threaded body portion within a threaded opening in an implant prior to the separating of the head portion of the break-off set screw from the threaded body portion.
